(19) United States
(12) Patent Application Publication
(10) Pub. No.: US 2002/0123676 A1
(43) Pub. Date: Sep. 5, 2002

(54) Measuring device for body fluids and infusion set and dialysis probe comprising such a measuring device one

(76) Inventors: Ulrich Haueter, Grosshoechstetten (CH); Ulrich Diermann, Ipsach (CH)

Correspondence Address: David E. Bruhn, DORSEY & WHITNEY LLP, Suite 1500, 50 South Sixth Street, Minneapolis, MN, 55402-1498, US

(21) Appl. No.: 10/087,522

(22) Filed: Mar. 1, 2002

Related U.S. Application Data
(63) Continuation of application No. PCT/CH00/00456, filed on Aug. 29, 2000.

(30) Foreign Application Priority Data
Sep. 8, 1999 (DE) DE 199 42 898 A1

(51) Int. Cl. A61B 5/00

(52) U.S. Cl. 600/309; 604/4.01

(57) ABSTRACT

The invention relates to a measuring device for body fluids, comprising an access (2) to the interior of the body and a sensor (1) to which the body fluid to be measured is supplied via the access (2). The sensor (1) is arranged on a part of the measuring device outside the body, in the immediate vicinity of the end, outside the body, of the access (2). The invention further relates to an infusion set and a dialysis probe comprising such a measuring device.

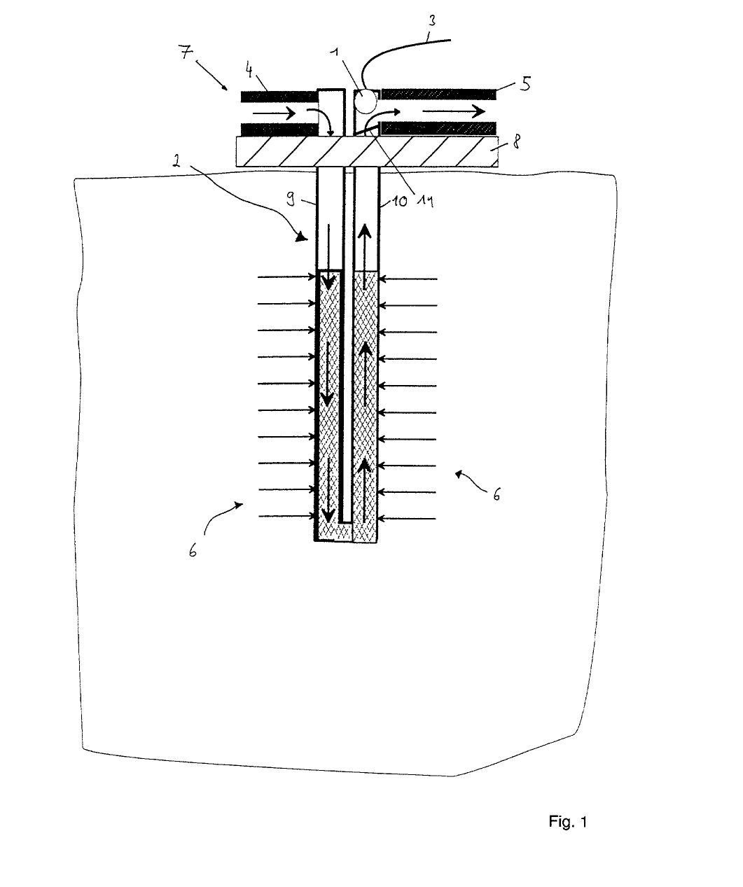

### BACKGROUND OF THE INVENTION

1. Technical Field

The present invention relates to a measuring device for body fluids in accordance with the preamble of patent claim 1, and an infusion set and a dialysis probe comprising such a measuring device.

2. Description of the Related Art

Such measuring devices serve in particular to detect the concentration or presence of substances in body fluids. A known way of performing such detecting, when determining blood sugar concentration, is to slit the patient's skin slightly, for example on the finger, and to ascertain with the aid of test strips the blood sugar content from the blood and/or intercellular fluid thus obtained. This method has the particular disadvantage that continuously detecting and measuring the values over a long period of time is not possible.

DE 37 42 263 A1 describes a means which accesses body fluids by means of a permanently implanted port body. Blood is sampled via said port body, wherein the port body can additionally be used as an infusion device. U.S. Pat. No. 4,955,861 also proposes injecting fluids into and/or sampling them from a patient's body by means of a permanent implant.

The disadvantage of the two methods above is that they do not guarantee an optimally exact measurement. Both cited documents propose that the body fluid is always sampled from the body before it is analysed. The volume to be analysed is then disadvantageously exposed to a change in temperature on the one hand, and on the other hand said conventional measurement disadvantageously runs discontinuously.

Furthermore, so-called body fluid sampling and dialysis systems are known in which the fluid to be measured is guided away from the body, for example via a hose, and up to a sensor. In this way, the sensor regularly lies far from the body. “Far” means that the sensor is 10 cm or more away from the sampling point. This causes a large time delay between changes in the physiological parameters and their detection. This time delay is caused by a long fluid column between the sampling point and the analysis point, this being dependent on the rate of delivery of the sample solution.

### SUMMARY OF THE INVENTION

It is the object of the present invention to provide a measuring device for body fluids which overcomes the above-cited disadvantages of the prior art. In particular, continuous monitoring is to be achieved with a high measuring accuracy, the aim being to detect the actually present values and changes in them in the shortest time possible.

This object is solved in accordance with the invention by arranging the sensor of the measuring device on a part of the measuring device lying outside the body in the immediate vicinity of the end, outside the body, of an access to the interior of the body. This advantageously guarantees in accordance with the invention that measuring takes place very near to the point in the interior of the body where the body fluid to be tested is actually present. Time delays in detecting changes in the readings are greatly shortened and there exists the possibility of reacting immediately to critical changes. A further advantage is that the sensor is accessible from without and can therefore be inserted, exchanged or removed by the patient himself, preferably only after the sterilisation process. Such an arrangement also has advantages with respect to the official admission of the device. Attaching the sensor externally also prevents the sensor exerting possible negative effects on the body.

In a preferred embodiment of the measuring device in accordance with the invention, the sensor is arranged such that it may be removed and/or exchanged, preferably pinned or clicked on. This makes handling the measuring device easier for the user, who in the present case will for the most part be the patient himself, and also makes it easier for the manufacturer to carry out necessary sterilising measures. This also circumvents the known problems of impairing the sensor through sterilisation. Particularly preferably, the sensor is arranged as near to the sampling point as is possible with a releasable attachment to the outlet end of the access of the measuring device.

In accordance with another preferred embodiment of the invention, there exists the possibility of arranging a valve means in the fluid channel which surrounds the fluid flow from the access to the sensor, said valve means preventing a reverse flow of the fluid from the sensor into the access. This ensures that the sensor is not free from the body fluid to be measured over a certain period of time, or in the case of a reverse flow, that no fluid is detected or that the same fluid is not detected repeatedly.

In accordance with an advantageous variant of the measuring device in accordance with the invention, the sensor can be arranged on a catheter head of an infusion set. Furthermore, there exists the possibility of arranging the sensor in the outlet, outside the body, of an implanted analysis probe.

Correspondingly, the invention also relates to an infusion set and a dialysis probe comprising a measuring device such as has been described above.

### DETAILED DESCRIPTION OF A PREFERRED EMBODIMENT

The present invention will now be explained in more detail by way of an embodiment and referring to the enclosed drawing. The drawing shows a dialysis probe comprising the measuring means in accordance with the invention.

The dialysis probe shown in the drawing comprises an access 2 to the interior of the body shown crosshatched, said access 2 comprising a supply tube 9 and a discharge tube 10. In this microdialysis probe, a dialysis fluid flows in the tubes 9 and 10 in the direction indicated by the arrows shown in said tubes 9 and 10. The fluid flow is diverted it its lower end directly by the tubes 9 and 10, which are connected to each other, and while flowing through these tubes picks up constituents from the surrounding tissue fluid, as shown by the arrows marked with the reference numeral 6.

The probe head of the dialysis probe, indicated as a whole by the reference numeral 7, lies outside the body. The probe is held via its two tubes 9 and 10 by a supporting plate 8 in the section where the two tubes emerge from the interior of the body. The inlet 4 for the dialysis probe, which is formed as a hose or a pipe and feeds into the tube 9, and the outlet 5, which can likewise be a hose or a tube and into which the tube 10 feeds from the interior of the body, are situated above said supporting plate 8.

Dialysis thus takes place in such a way that dialysis fluid is introduced into the tube 9 in the interior of the body via the inlet 4. While flowing through the tubes 9 and 10, which are surrounded by a membrane, the dialysis fluid picks up substances from the surrounding tissue, and then leaves the dialysis probe through the outlet 5.

A sensor 1 is then arranged in accordance with the invention on the upper part of the tube 10, outside the body, just before the entrance of the fluid into the outlet 5, said sensor 1 being able for example to detect blood sugar concentration. Said sensor can of course also be suitable for other readings to be detected in body fluids. The sensor is connected to a measuring cable which leads to an evaluation unit (not shown).

In accordance with the invention, the sensor 1 is attached in the immediate vicinity of the emergence of the tube 10 from the body, and so can detect changes in the composition of the fluid in the tube 10 very quickly. Furthermore, it is advantageously attached outside the body directly to the emerging end of the tube 10, such that it is accessible from without and can be manipulated by the user himself. These manipulations relate for example to inserting, exchanging or sterilising the sensor 1. In its position outside the body, it furthermore advantageously does not come into direct contact with the body and cannot therefore exert a negative influence, for example by contamination.

A reflux valve 11 is also schematically shown, above the supporting plate 8 in the upper part of the tube 10, said valve in this case being able to prevent dialysis fluid from flowing in the wrong direction, i.e. back into the tube 10. This reflux valve or a similar means in its place can also be supplemented by a means which is accessible from without and which seals off the tube 10 at this position, so that the sensor 1 can be exchanged once the flow of the dialysis fluid has been stopped.

In approximately the same way as in the example embodiment shown in the drawing, it is likewise possible to arrange a sensor in the immediate vicinity of the end, emerging from the body, of an infusion set catheter needle. If this needle comprises only one flow channel, fluid can then be suctioned briefly from the interior of the body via this channel and analysed by the sensor.

In the foregoing description preferred embodiments of the invention have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

what is claimed is:

1. A measuring device for body fluids, comprising an access (2) to the interior of the body and a sensor (1) to which the body fluid to be measured is supplied via said access (2), characterised in that said sensor (1) is arranged on a part of said measuring device outside the body, in the immediate vicinity of the end, outside the body, of said access (2).

2. The measuring device as set forth in claim 1, characterised in that said sensor (1) is arranged such that it may be removed and/or exchanged.

3. The measuring device as set forth in claim 1, characterised in that a valve means is arranged in the fluid channel which surrounds the fluid flow from said access (2) to said sensor (1), said valve means preventing a reverse flow of the fluid from said sensor (1) into said access (2).

4. The measuring device as set forth in claim 1, characterised in that said sensor (1) is arranged on a catheter head (7) of an infusion set.

5. The measuring device as set forth in claim 1, characterised in that said sensor (1) is arranged in the outlet (5), outside the body, of an implanted dialysis probe.

6. An infusion set comprising a measuring device as set forth in claim 1.

7. A dialysis probe comprising a measuring device as set forth in claim 1.
